(19) United States
(12) Patent Application Publication
(10) Pub. No.: US 2008/0311231 A1
(43) Pub. Date: Dec. 18, 2008

(54) Broad Spectrum Non-Traditional Preservative System

(76) Inventors: Shanta M. Modak, Riveredge, NJ (US); Lauserpina A. Caraos, Hollis, NY (US)

Correspondence Address: BAKER BOTTS L.L.P., 30 ROCKEFELLER PLAZA, 44TH FLOOR, NEW YORK, NY, 10112-4498, US

(21) Appl. No.: 12/136,530

(22) Filed: Jun. 10, 2008

Related U.S. Application Data
(63) Continuation of application No. PCT/US06/61927, filed on Dec. 12, 2006.
(60) Provisional application No. 60/749,440, filed on Dec. 12, 2005.

(51) Int. Cl. A61K 36/53; A61K 36/00; A61K 36/48; A61K 31/191; A61P 31/04

(52) U.S. Cl. 424/745; 424/725; 424/757; 514/574

(57) ABSTRACT

Preservative compositions containing emollient solvents such as octanediol or Symdiol™ or octoxyglycerine monoesters in combination with organic acids such as alpha hydroxyl acids can be used for preventing microbial growth and spoilage in cosmetic and topical skin formulations with the added benefit of providing silky smooth texture to skin. The compositions may optionally contain an essential oil/component. These preservative compositions are odorless and colorless and small concentrations can be added to cosmetic/topical formulations to prevent bacterial, yeast and fungal growth within 24 to 48 hours after contamination.

### CROSS-REFERENCE TO RELATED APPLICATIONS

This application claims the benefit of U.S. provisional application Ser. No. 60/749,440 filed Dec. 12, 2005 of which is incorporated by reference in its entirety herein.

### 1. FIELD OF THE INVENTION

The present invention provides broad spectrum preservative compositions comprising combinations of an emollient solvent and an alpha-hydroxy acid, and optionally, one or more essential oil components. These compositions, while having a broad spectrum antimicrobial activity, also possess hypo-allergenic properties making them suitable for use in topical and cosmetic formulations. In specific embodiments of the present invention, preservative compositions containing emollient solvents such as octanediol, Symdiol™ or Sensiva™ (octoxy glycerine or ethyl hexyl glycerine) or monoesters in combination with alpha hydroxy acids, excluding metal salts of these acids, can be used for preventing microbial growth and spoilage in cosmetic and topical skin formulations.

### 2. BACKGROUND OF THE INVENTION

Preservatives are routinely used in topical formulations and in cosmetics to prevent bacterial and fungal growth that cause odor and discoloration. Commonly used preservatives include antibacterial agents such as quaternary ammonium compounds, chlorinated phenols, parabens, imidazolidinyl urea, phenoxy ethanol, benzoic acid, and sorbic acid. Formaldehyde-releasers and isothiazolinones may also be used. These compounds, however, are not adequate either due to lack of broad spectrum activity or production of allergic reactions in a large number of individuals. Essential oils and botanicals are also used as preservatives, however these ingredients add color and odor to cosmetics and may also cause dermal irritation. Some parameters used in testing a preservative include antimicrobial efficacy, stability, induction of cutaneous adverse reactions and reported rates of sensitization.

Organic acids currently find use in bakery products to improve shelf life without affecting volume, appearance, taste or flavor of the product. The organic acids used most frequently in bakery products include sorbic and propionic acids. A sub-group of organic acids namely alpha hydroxy acids (“AHA”) ranging from citric, lactic, glycolic, mandelic, malic and tartaric acid may also be used to prevent microbial activity and enhance the preservation of cosmetic and topical products. AHAs are marketed as having “anti-aging” and “rejuvenating” properties.

Certain emollient solvents exhibit synergistic action when combined with essential oils or ingredients against microorganisms as noted in U.S. patent application Ser. No. 10/785,207, filed Feb. 24, 2004, which is incorporated by reference in its entirety. The emollient solvents used as preservatives in cosmetics do not usually produce skin reactions, and in addition, render the skin smooth and silky. However, the antibacterial action of these compounds is limited frequently to only gram positive organisms.

There is therefore a need in the art for a non-traditional preservative system which is gentle and non-irritating. The compositions of the invention may be incorporated into creams, lotions, and cosmetics or other compositions to be used in daily care, health care or infant care.

### 3. SUMMARY OF THE INVENTION

The present invention relates to a combination of AHA and emollient solvent formulations which are more effective than prior art compositions in acting as antimicrobial preservatives while decreasing the likelihood of an allergic response in the skin of the user.

Specifically, the present invention is directed to a topical composition comprising a preservative composition wherein the preservative composition comprises a combination of emollient solvent and an alpha hydroxy acid, and wherein the combination shows greater than additive antimicrobial activity. In certain embodiments, the topical composition may also contain an essential oil component.

In certain embodiments, the combination of emollient solvent and alpha hydroxy acid shows greater than additive antimicrobial activity in an amount effective in reducing or preventing the growth of microorganisms. In other embodiments, the combination of emollient solvent and alpha hydroxy acid shows greater than additive antimocrobial activity in an amount having broad spectrum antimicrobial activity.

In certain embodiments, the broad spectrum antimicrobial activity comprises activity against bacteria, yeast-like fingi or non-yeast fungi or viruses.

In specific embodiments, the emollient solvent is selected from the group consisting of octanediol, hexanediol, Symdiol™, Sensiva™ (octoxy glycerin), propylene hepanoate, propylene caprylate and glyceryl caprylate. In other embodiments, the alpha hydroxy organic acid is selected from the group consisting of citric, lactic, benzoic, glycolic, mandelic, malic and tartaric acid.

The present invention is also directed to a topical composition comprising a antimicrobial preservative composition wherein the composition consists of a synergistic combination of one or more emollient solvent and an alpha hydroxy acid.

### 4. DETAILED DESCRIPTION OF THE INVENTION

For clarity, and not by way of limitation, the detailed description of the invention is divided into the following subsections:

(i) Compositions and components of cosmetic or other topical formulations according to the invention;

(ii) Methods of preparation and formulations of the preservative compositions; and

(iii) Methods of evaluation of the antimicrobial efficacy of the preservative compositions.

The present invention provides for broad spectrum antimicrobial preservative compositions comprising combinations of an emollient solvent and AHA, and optionally, an essential oil ingredient. In preferred non-limiting embodiments of the invention, such compositions contain emollient solvents such as octanediol, Symdiol™ or Sensiva™ or monoesters in combination with AHAs (excluding metal salts of these acids).

In order to prevent microbial growth in creams and other cosmetics without using traditional preservatives, the inventors of the present invention evaluated various emollients and other agents such as humectants, either alone or in combination, to determine whether these agents can prevent bacterial growth if used in higher concentrations. The present inventors evaluated emollient solvents alone or in combination with humectants such as alpha-hydroxy acids (AHA), which are commonly used in cosmetics or topical creams. Thus, the compositions of the present invention contain combinations of certain AHAs and emollient solvents, which were incorporated into a cream base and tested for their broad spectrum preservative action. It was unexpectedly discovered that the acids in combination with emollient solvent at low concentrations exhibit synergistic action against bacteria and yeast.

The phrase “pharmaceutically acceptable” as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

The terms “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. The term “amount” as used herein refers to quantity or to concentration as appropriate to the context. The effective amount of a drug that constitutes a therapeutically effective amount varies according to factors such as the potency of the particular drug, the route of administration of the formulation, and the mechanical system used to administer the formulation. A therapeutically effective amount of a particular drug can be selected by those of ordinary skill in the art with due consideration of such factors.

As used herein, the terms “non-traditional preservative” refers to the fact that the primary components of the present invention are not traditional preservatives. Rather, the components are compounds which have an antimicrobial effect, and therefore act as a type of preservative in an unconventional manner.

### 4.1 Compositions and Components of Cosmetic or Other Topical Formulations According to the Invention

Emollient solvents which may be used according to the invention include, but are not limited to one or more than one glycidyl ethers having alkyl chains up to and including 18 carbon molecules and ethoxylates and propoxylates thereof, glyceryl ethers having alkyl chains up to and including 18 carbon molecules and ethoxylates and propoxylates thereof, mono- and diglyceryl ethers having alkyl chains up to and including 18 carbon molecules and ethoxylates and propoxylates thereof, ethoxy diglycol esters, ethyl hexyl alcohol propoxylate, and propylene glycol ester ethoxylates and propoxylates, and preferably Arlarnol™ (Altas). Specific examples of emollient solvents include but are not limited to octanediol, hexanediol, Symdiol™ (a combination of octanediol and hexanediol), and octoxyglycerine, monoesters such as propylene hepanoate, propylene caprylate, glyceryl caprylate, propylene glycol, and butylene glycol. Intermediary stock solutions of emollient solvents can range between about 30 to about 60% w/w, preferably from about 45 to about 55% w/w, and most preferably are about 50% w/w. However, in certain embodiments, the stock solution ranges from about 50% to about 99% w/w. A suitable final concentration of emollient solvent in a cosmetic or topical preparation is between about 0.05 to about 5% w/w, preferably between about 0.05 to about 1% w/w, preferably between about 0.15 to about 0.8% w/w, and most preferably between about 0.2 to about 0.5% w/w. However, in certain embodiments, the emollient solvent ranges from about 0.3% to about 5% w/w.

AHA which may be used according to the invention include, but are not limited to, various AHAs such as glycolic, lactic, citric, mandelic, malic and tartaric acid however, metal salts of these salts are excluded). In preferred non-limiting embodiments, metal salts of these AHAs are excluded. Intermediary stock solutions of an AHA or other organic acid can range between about 1 to about 50% w/w, preferably from about 5 to about 30% w/w, more preferably about 15 to about 25% w/w, and most preferably about 20% w/w. A suitable final concentration of an AHA in a cosmetic or topical preparation may be between about 0.01% to about 5.0% w/w, preferably 0.05% to about 3% w/w, preferably about 0.05% and about 1% w/w, more preferably between about 0.07 and about 0.75% w/w, and most preferably between about 0.1 and about 0.5% w/w.

In certain embodiments of the invention, essential oils are used in combination with emollient solvents and AHAs. Essential oils (“EOs”), as defined herein, are volatile oils obtained from plant or animal sources, or their synthetic equivalents, and are composed of complex mixtures of several constituents as monoterpenes and sesquiterpene hydrocarbons, monoterpene and sesquiterpene alcohols, esters, ethers, aldehydes, ketones, oxides and the like. Examples of EOs include but are not limited to: bergamot oil, clary sage oil, sage oil, almond oil, ylang-ylang oil, neroli oil, sandalwood oil, frankincense oil, ginger oil, peppermint oil, lavender oil, jasmine absolute, geranium oil bourbon, spearmint oil, clove oil, patchouli oil, rosemary oil, rosewood oil, sandalwood oil, tea tree oil, vanilla oil, lemongrass oil, cedarwood oil, balsam oils, tangerine oil, Hinoki oil, Hiba oil, ginko oil, eucalyptus oil, lemon oil, orange oil, thyme oil, savory oil, oregano oil, and sweet orange oil. Botanicals, such as camphor and cinnamon may also be used. Individual constituents (“ICs”) of essential oils may be natural or entirely or partially synthetic, and include, but are not limited to, 1-citronellol, α-amylcinnamaldehyde, lyral, geraniol, farnesol, hydroxycitronellal, isoeugenol, eugenol, eucalyptol, linalool, citral, thymol, limonene and menthol. Additionally, sesquiterpenoids such as nerolidol, farnesol, bisabolol and apritone may also be used in the present invention. Mixtures of one or more EO, one or more IC, and one or more EO as well as one or more IC, are encompassed by the present invention. Preferably, the essential oil component is selected from the group consisting of thyme oil, savory oil, oregano oil, rosewood oil, lavendar oil, basil oil, farnesol, and bisbolol. The concentrations of these EOs and ICs may be between about 0.01 and about 10 percent; preferably between about 0.05 and about 1.0 percent or between about 0.05 and about 0.5 percent, and more preferably between about 0.2 and about 0.4 percent. In preferred embodiments, the EO is lemon oil and/or the IC is farnesol.

In certain embodiments of the claimed invention, no additional preservatives (traditional or nontraditional) are included. Specifically, traditional preservatives not to be included in these embodiments of the present invention include but are not limited to chlorhexidine and derivative thereof such as chlorhexidine palmitate, chlorhexidine diphosphanilate, chlorhexidine digluconate, chlorhexidine diacetate, chlorhexidine dihydrochloride, etc., quaternary ammonium compounds, iodopropynylbutyl carbamate (IPBC; Germall plus). benzoic acid, dehydroacetic acid, propionic acid, sorbic acid, methyl paraben, ethyl paraben, propyl paraben, butyl paraben, cetrimide, benzalkonium chloride, benzyl alcohol, bronopol, chlorbutanol, ethanol, phenoxyethanol, phenylethyl alcohol, 2,4-dichlorobenzyl alcohol, thiomersal, and chlorinated phenols. Specifically, nontraditional preservatives not to be included in these embodiments of the present invention include but are not limited to agents with, anti-viral, viristatic and/or viricidal, anti-bacterial, bacteriocidal and/or bacteriostatic, anti-fungal, fungicidal and/or fungistatic activities, and combinations thereof. Examples of such antimicrobial preservative agents include, but are not limited to, chlorhexidine gluconate (CHG), or benzalkonium chloride (BZK). Specific additional examples of such anti-microbial agents include, but are not limited to, iodophors, iodine, dequalinium chloride, chlorhexidine, chloroeresol, chlorxylenol, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, and pharmaceutically acceptable salts thereof. These and further examples of anti-microbial agents can be found in such references as Goodman and Gilman's The Pharmacological Basis of Therapeutics (Goodman Gilman A, Rall T W, Nies A S, Taylor P, ed. (Pergamon Press; Elmsford, N.Y.: 1990)), the contents of which are hereby incorporated by reference. Essential oils also may be considered as anti-microbial compounds under certain circumstances and are included in the above list of anti-microbial compounds not included in the present invention.

In another aspect of the invention, in certain embodiments, one or more traditional or nontraditional preservatives may be added to the compositions of the invention. In various non-limiting embodiments, an additional traditional preservative (not included in the compositions of the claimed invention) may be included in the compositions of the claimed invention. These additional preservatives may be any one or more of the following antiseptics, and combinations thereof, including but not limited to chlorhexidine and derivative thereof such as chlorhexidine palmitate, chlorhexidine diphosphanilate, chlorhexidine digluconate, chlorhexidine diacetate, chlorhexidine dihydrochloride, etc., quaternary ammonium compounds, iodopropynylbutyl carbamate (IPBC; Germall plus). benzoic acid, dehydroacetic acid, propionic acid, sorbic acid, methyl paraben, ethyl paraben, propyl paraben, butyl paraben, cetrimide, benzalkonium chloride, benzyl alcohol, bronopol, chlorbutanol, ethanol, phenoxyethanol, phenylethyl alcohol, 2,4-dichlorobenzyl alcohol, thiomersal, and chlorinated phenols.

In various non-limiting embodiments, an added non-traditional preservative (not included in the compositions of the claimed invention) may be any one or more of the following agents, in amounts sufficient to produce a preservative/antimicrobial effect, including, but not limited to agents with, anti-viral, viristatic and/or viricidal, anti-bacterial, bacteriocidal and/or bacteriostatic, anti-fungal, fungicidal and/or fungistatic activities, and combinations thereof. Examples of such antimicrobial preservative agents include, but are not limited to, chlorhexidine gluconate (CHG), or benzalkonium chloride (BZK). Specific additional examples of such anti-microbial agents include, but are not limited to, iodophors, iodine, dequalinium chloride, chlorhexidine, chloroeresol, chlorxylenol, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, and pharmaceutically acceptable salts thereof. These and further examples of anti-microbial agents can be found in such references as Goodman and Gilman's The Pharmacological Basis of Therapeutics (Goodman Gilman A, Rall T W, Nies A S, Taylor P, ed. (Pergamon Press; Elmsford, N.Y.: 1990)), the contents of which are hereby incorporated by reference. Essential oils also may be considered as anti-microbial compounds under certain circumstances and are included in the above list of anti-microbial compounds not included in the present invention.

A cream base according to the invention may comprise but is not limited to a formulation of Crodalan AWS™ (for example about 2.0 percent w/w), Crodacol S-70™ (for example about 3.0 percent w/w), mineral oil (for example about 9.5 percent w/w), propylene glycol (for example about 2.0 percent w/w) and water (for example about 83.5 percent w/w).

Ancillary components, whose use is optional, impart additional desirable properties to the composition of the present invention. These components can include but are not limited to a lipid-soluble component; an emulsifier component; an antioxidant component; a solvent component; a thickener component; and a hydrophilic component. Minor adjunct ingredients may be present in the cosmetic compositions. Among them may be the water-soluble vitamins, colorants, fragrances and botanicals.

The lipid-soluble component can comprise at least one ingredient selected from the group consisting of: (1) dimethicone; (2) bisabolol; (3) polyoxyethylene fatty acid esters; (4) cetyl alcohol; (5) a glyceryl triester of a medium-chain carboxylic acid selected from the group consisting of tricaproin, tricaprylin, tricaprin, and mixtures thereof; (6) white petrolatum; and (7) mineral oil.

Emulsifiers serve two functions. They act like a solubilizing agent to combine the water-soluble and non-water-soluble phases together; that is, to form a stable bridge between the waters and the oils of the ingredients. The emulsifiers also serve as emollients, providing a pleasant, esthetically appropriate tactile feeling when the emulsified composition is applied to the skin. The emulsifier component is present in a quantity sufficient to combine water-soluble and non-water-soluble phases of the composition. The emulsifier component can comprise at least one of a mixture of mono- and distearate esters of polyoxyethylene and free polyethylene oxide, partial esters of lauric, palmitic, stearic, and oleic acids and hexitol anhydrides, and 120-mole ethoxylated jojoba oil. Preferably, the emulsifier component comprises a mixture of mono- and distearate esters of polyoxyethylene and free polyethylene oxide, partial esters of lauric, palmitic, stearic, and oleic acids and hexitol anhydrides and 120-mole ethoxylated jojoba oil.

Ambient temperature is defined herein between about 20 and about 35° C. Room temperature is defined herein between about 20 and about 25° C.

Specific, non-limiting embodiments of the invention including the following compositions may further comprise additional ingredients that do not substantially affect the antimicrobial properties of the composition. For the following formulations, water, where indicated, was added last to the other ingredients to bring the total volume to 100 percent.

### 4.2 Method of Preparation and Formulations of the Preservative Compositions

The present invention provides for the preparation of a cream base comprising an emollient solvent or AHA. A cream base according to formulations known in the art, or as described below, may be used.

In one non-limiting example, the invention provides for a cream base comprising: water between about 80 to about 90% w/w, preferably between about 80 to about 85% w/w and most preferably between about 83 to about 84% w/w; propylene glycol between about 2 to about 5% w/w, preferably between about 2 to about 4% w/w, and most preferably about 2% w/w; mineral oil between about 9 to about 12% w/w, preferably between about 8 to about 9% w/w, and most preferably about 9.5% w/w; Crodacol S-70™ between about 2 to about 4% w/w, preferably between about 2.5 to about 3.5% w/w and most preferably about 3% w/w; Crodalan AWS™ between about 1 to about 3% ww, preferably between about 1.5 to about 2.5% w/w, and most preferably about 2% w/w.

The preservative in the above cream base may, for example, include any one of the following additional components: (i) an emollient solvent as set forth above and present in an amount between about 0.1 to about 0.7% w/w such as; octanediol between about 0.1 to about 0.7%, preferably between about 0.4 to about 0.5%, and most preferably about 0.5% w/w; or Symdiol™ between about 0.1 to about 0.7%, preferably between about 0.4 to about 0.5%, and most preferably about 0.5% w/w; or Sensiva™ between about 0.1 and about 0.7% preferably between about 0.4 to about 0.5%, and most preferably about 0.5% w/w; and (ii) an AHA as set forth above and present in an amount between about 0.05 to about 0.5% w/w, preferably between about 0.1 to about 0.3% w/w, and most preferably about 0.2% w/w; such as lactic acid between about 0.05 to about 0.5% w/w, preferably between about 0.1 to about 0.3% w/w, and most preferably about 0.2% w/w; citric acid between about 0.05 to about 0.4% w/w, preferably between about 0.1 to about 0.3% w/w, and most preferably about 0.2% w/w; or mandelic acid between about 0.05 to about 0.4% w/w, preferably between about 0.1 to about 0.3% w/w, and most preferably about 0.2% w/w. Additionally, a mixture of AHAs and/or emollient solvents, together producing the above-listed amounts, may be used. Accordingly, the present invention also provides for the preparation of a cream base prepared according to a formulation which comprises but is not limited to a combination of two emollient solvents and one organic acid, two organic acids and one emollient solvent or additional combinations thereof.

In another non-limiting example, the present invention provides for a cream base composition comprising: (i) water between about 80 to about 90% w/w, preferably between about 80 to about 85% w/w, and most preferably between about 83 to about 84% w/w; propylene glycol between about 2 to about 5% w/w, preferably between about 2 to about 4% w/w, and most preferably about 2% w/w; mineral oil between about 9 to about 12% w/w, preferably between about 8 to about 9% w/w, and most preferably about 9.5% w/w; Crodacol S-70™ between about 2 to about 4% w/w, preferably between about 2.5 to about 3.5% w/w, and most preferably about 3% w/w; Crodalan AWS™ between about 1 to about 3% w/w, preferably between about 1.5 to about 2.5% w/w, and most preferably about 2% w/w; and (ii) a preservative comprising any two or more of the following additional components from the list of emollient solvents and AHAs: an emollient solvent in an amount between about 0.1 and about 0.7% w/w such as; octanediol between about 0.1 to about 0.7% w/w, preferably between about 0.4 to about 0.5% w/w, and most preferably about 0.5% w/w; Symdiol™ between about 0.1 to about 0.7% w/w, preferably between about 0.4 to about 0.5% w/w, and most preferably about 0.5% w/w; an AHA in an amount between about 0.05 to about 0.4% w/w, preferably between about 0.1 to about 0.3% w/w, and most preferably about 0.2% w/w; lactic acid between about 0.05 to about 0.4% w/w, preferably between about 0.1 to about 0.3% w/w, and most preferably about 0.2% w/w; citric acid between about 0.05 to about 0.4%, preferably between about 0.1 to about 0.3%, and most preferably about 0.2%; mandelic acid between about 0.05 to about 0.4% w/w, preferably between about 0.1 to about 0.3% w/w, and most preferably 0.2% w/w.

A preservative according to the invention may be added directly to a cream base to obtain the desired final concentration as set forth above or may be prepared in an intermediate step as an intermediate stock solution which comprises a pre-determined dilution of an emollient solvent, AHA or combinations thereof as described in Example 7. Thus, an intermediate stock preservative composition, for example may comprise between about 30 to about 50% w/w octanediol, about 30 to 5 about 0% w/w octanediol and about 10 to about 50% w/w lactic acid, about 30% to about 50% w/w Symdiol™, about 30% to about 50% w/w Symdiol™ and about 10 to about 20% w/w lactic acid, about 30 to about 50% w/w Sensiva™ and about 10 to about 50% lactic acid. The remainder of the intermediate stock composition being made up to 100% by addition of an appropriate solvent such as water, propylene glycol, alcohol, etc.

### 4.3 Method of Evaluation of the Antimicrobial Efficacy of the Preservative Compositions:

The invention provides for a method to evaluate the antimicrobial activity of a preservative composition. Evaluation may be performed by, but is not limited to measuring the survival, viability, or growth rate of a test microorganism when exposed to a preservative formulation of the invention. The invention provides for a method which measures the growth rate, and relative inhibition of growth of a test microorganism population compared to a control, mock treated or untreated population of a test microorganism. The method of evaluation may be performed using a physical, biochemical or biological means of determining the number of surviving cells in a test population at the start and end point of the evaluation. The method of evaluation may be based on enumerating single cells or by a colony count method. The invention provides for a physical method such as a total cell count or a viable cell count. The method of evaluation of a preservative may be performed by either a macroscopic or microscopic method. The method of evaluation may thus utilize specialized instruments such as a microscope, a microtiter plate reader, spectrophotometer or fluorescence activated cell sorter. The evaluation of efficacy of a preservative effective against a microorganisms, as used herein, includes, but is not limited to evaluation in controlling the growth of bacteria, (both gram-positive and -negative), fungi, algae, viruses, amoebae, spores, and the like, and include both yeast-like fungi and mold-like fungi.

In a non-limiting example, about 500 μl or about 250 μl of a preservative formulation is added to about 49.5 or about 49.75 gm respectively of a cream base to obtain the desired concentrations for testing. The preservative formulation and cream base is mixed to homogenously distribute all components. About 1 gm quantity of formulation is dispensed into several culture tubes and each tube inoculated with 10 μl of culture containing about 10⁵ or about 100 μl of culture containing about 10⁷ cfu (colony forming units) of test organism. The tubes are incubated at room temperature or at about 37° C. for bacterial or yeast cultures or at about 30° C. for yeast or fungal cultures with subculturing on Sabouraud agar. After about 24 hours, the tubes are removed and about 9 ml of drug inactivating medium is added and mixed. After making another dilution (1:10) with drug inactivating medium, a 0.5 ml aliquot is subcultured on to a TSA plate. Colony counts are measured after 24 hours incubation at 37° C. or other appropriate temperature to determine the efficacy of a preservative.

According to the present invention, effective activity of a preservative is one that is capable of controlling the growth of microorganisms in a preparation. Preservative activity means to inhibit microbial, primarily bacterial or fungal growth. Preservative activity includes both the reduction and/or prevention of such growth. Preservative activity includes maintaining a microorganism population at a desired level including undetectable levels such as zero population, reducing a microorganism population to a desired level, and/or inhibiting or slowing the growth of microorganisms that are initially present or may enter a topical formulation or cosmetic during the course of normal use. The present invention also provides a method for preserving a topical formulation or cosmetic susceptible to spoilage by microorganisms comprising the steps of adding to the material or medium a preservative composition of the present invention in an amount effective to control the growth of the microorganism.

In non-limiting embodiments, the present invention provides for the evaluation of a preservative composition in controlling the growth of microorganisms classified as bacteria. This classification includes both gram-positive and -negative bacterial species, including but not limited to Escherichia coli, Salmonella spp., Bacillus spp., Klebsiella spp., Bordetella spp. Clostridium spp., Mycobacterium spp., Enterococcus spp., Lactobacillus spp., Borrelia spp., etc. The present invention also provides for evaluation of a preservative composition against growth of other less commonly known or encountered microorganisms such as Actinomycetes spp., Bacteroides spp., Chlamydia spp., Azotobacter spp., Cyanobacteria, Campylobacter, etc.

In other non-limiting embodiments the present invention provides for the evaluation of a preservative composition in controlling the growth of microorganisms classified as fungi. While over 60,000 species of fungi are known, the present invention preferably tests for activity against fungal species commonly associated with spoilage of cosmetic preparations including but not limited to unicellular fungi such as the yeast Saccharomyces spp., or multicellular fungi such as Penicillium spp.

In non-limiting embodiments, evaluation of the activity of specific combinations of emollient solvent and AHA may be performed to determine efficacy of preservative activity and/or effectiveness against a specific microorganism or spectrum of organisms including but not limited to bacteria and fungi. A specific combination of emollient solvent and AHA may show enhanced or synergistic activity. The preservative activity of a combination of an emollient solvent and AHA showing enhanced or synergistic activity will at a minimum exceed the preservative activity of each individual component utilized alone, and may exceed the sum of antimicrobial activity demonstrated by each individual component, if the combination is synergistic.

The present invention provides for a preservative combination comprising an emollient solvent such as octanediol or Symdiol™ used at a concentration of between about 0.1 to about 0.5% w/w, preferably about 0.25 to about 0.35% w/w, and most preferably at about 0.3% w/w, and an AHA including but not restricted to citric acid, lactic acid or mandelic acid used at a concentration of between about 0.1 to about 0.5% w/w, preferably about 0.15 to about 0.25% w/w, and most preferably at about 0.2% w/w. Use of a combination of emollient solvent and AHA according to the present invention may result in an additive or greater than additive effect of the preservative action, than similar concentrations of each preservative alone. Thus for example about 0.3% w/w octanediol, about 0.3% w/w Symdiol™ or about 0.25% w/w lactic acid may each result in a two order of magnitude reduction of a contaminating microorganism e.g. lowering survival of a test organism to about 10⁶ CFU from an initial count of about 10⁸ CFU. An additive effect would result in a net reduction to 2×10⁶ CFU of surviving microorganisms. A synergistic combination would result in a reduction of between three to eight orders of magnitude i.e. 10⁵ to 0 CFU. See Examples 4 and 6 for octanediol and AHA combinations and Example 5 for Symdiol™ and AHA combinations.

### 5. WORKING EXAMPLES

The following examples are by way of example, not by way of limitation, of the principles of the present invention, to illustrate the best mode of carrying out the invention.

In order to develop a broad spectrum hypo-allergenic preservative system for topical and cosmetic formulations, emollient solvents alone or in combination with certain organic acids which are commonly used in cosmetics; or combination of two or more emollient solvents, were evaluated. The organic acids selected were alpha hydroxy acids ranging from citric, lactic, glycolic, lactic, mandelic, malic and tartaric acid (metal salts of these acids are excluded). The emollient solvents include octanediol, hexanediol, Symdiol™, octoxy glycerine, monoesters such as propylene hepanoate, propylene caprylate, glyceryl caprylate etc.

Preservative compositions of the present invention containing combinations of AHA and emollient solvent were incorporated into a cream base and tested for their broad spectrum preservative action. Preservative compositions prepared according to the present invention and containing combinations of AHA and emollient solvents, exhibited greater that additive anti-bacterial, anti-yeast and anti-fungal activity compared to the activity shown by each component when used alone.

### Example 1

### Cream Based Formulation

The following cream based formulation was prepared.

TABLE 1
Cream base formulation

| Ingredient | % w/w |
|---|---|
| Water | 83.5 |
| Propylene glycol | 2.0 |
| Mineral oil | 9.5 |
| Crodacol S-70 ™ | 3.0 |
| Crodalan A WS ™ | 2.0 |

### Example 2

### Preservative Compositions

The following preservative compositions were prepared (% w/w).

TABLE 2

| Emollient Solvent | AHA | Other |
|---|---|---|
| 50% Octanediol | 20% Mandelic acid | 30% water |
| 50% Octanediol | 20% Lactic acid | 30% water |
| 50% Octanediol | 20% Citric acid | 30% water |
| 50% Symdiol ™ | 20% Citric acid | 30% water |
| 50% Symdiol ™ | 20% Lactic acid | 30% water |
| 50% Symdiol ™ | 20% Mandelic acid | 30% water |

### Example 3

### Evaluation of Antimicrobial Spectrum of Various Preservative Compositions

Method of evaluation—Method A: 500 μl or 250 μl of each of the following preservatives was added to 49.5/49.75 gm of the cream base to obtain the desired concentrations for testing as in Table 3, and mixed well. 1.0 gm of this base+preservative formulation was dispensed into several culture tubes and each tube was inoculated with 10 μl of culture containing 10⁵ cfu (colony forming units) of test organism. The tubes were incubated at room temperature. After 24 hours the tubes were removed and 9 ml of drug inactivating medium was added and mixed. After making another dilution (1:10) with drug inactivating medium, a 0.5 ml aliquot was subcultured on to a TSA plate. Colony counts were measured after 24 hours incubation at 37° C.

TABLE 3

| Emollient Solvent (% w/w) | AHA (% w/w) | Other (% w/w) |
|---|---|---|
| 50% Octanediol | 20% Mandelic acid | 30% water |
| 50% Octanediol | 20% Lactic acid | 30% water |
| 50% Octanediol | 20% Citric acid | 30% water |
| 50% Symdiol ™ | 20% Citric acid | 30% water |
| 50% Symdiol ™ | 20% Lactic acid | 30% water |
| 50% Symdiol ™ | 20% Mandelic acid | 30% water |

Result: Preservative activity of various creams utilizing a concentration of test organism of 10⁵ cfu/gm and incubation at room temperature is shown in Table 4.

TABLE 4

| Conc. of ingredients in the | Colony counts (CFU)/gm | | |
|---|---|---|---|
| cream (% w/w) | C. albicans | E. coli | S. aureus |
| Control (Cream Base + culture) | 3.3 × 10⁵ | 2.5 × 10⁶ | 8 × 10⁵ |
| 0.5% Octanediol | 5.2 × 10² | 0 | 1.5 × 10³ |
| 0.3% Octanediol | 2.0 × 10⁵ | 5.4 × 10⁴ | 4.0 × 10⁴ |
| 0.5% Symdiol ™ | 4.5 × 10⁴ | 1.0 × 10⁵ | 2.1 × 10⁴ |
| 0.2% Lactic acid | 1.5 × 10⁴ | 2.3 × 10⁴ | 3.0 × 10³ |
| 0.2% Citric acid | 1.0 × 10⁵ | 7.3 × 10⁴ | 5.0 × 10⁴ |
| 0.5% Octanediol + 0.2% Lactic | 0 | 0 | 0 |
| acid | | | |
| 0.3% Octanediol + 0.1% Lactic | 0 | 0 | 0 |
| acid | | | |
| 0.5% Octanediol + 0.2% Citric | 0 | 0 | 3.0 × 10¹ |
| acid | | | |
| 0.3% Octanediol + 0.2% Citric | 2.0 × 10³ | 0 | 1.0 × 10³ |
| acid | | | |
| 0.5% Symdiol ™ + 0.2% Lactic | 0 | 0 | 0 |
| acid | | | |
| 0.5% Symdiol ™ + 0.2% | 0 | 0 | 0 |
| Mandelic acid | | | |
| 0.5% Symdiol ™ + 0.2% Citric | 0 | 0 | 0 |
| acid | | | |

Conclusion: Octanediol alone showed better efficacy than Symdiol™ alone against the bacteria tested. Both Octanediol and Symdiol™ exhibited enhanced activity with the AHAs tested especially against C. albicans and S. aureus. In the case of E. coli, enhanced activity can be seen with both Octanediol and Symdiol™.

### Example 4

### Evaluation of the Synergistic Activity of Octanediol and AHA Combinations Against Higher Concentrations of P. aeruginosa

Evaluation of the synergistic activity of octanediol and AHA combinations against higher concentrations of P. aeruginosa. Since 0.5% Octanediol kills 100% of P. aeruginosa at a concentration of 105 cfu/gm, the test was repeated with this organism at 107 cfu/gm.

Method of evaluation: Same as described in Method A except a higher inoculum was used. A 100 μl inoculum containing 10⁷ cfu of P. aeruginosa was added to each 1 gm of cream and incubated at 37° C.

TABLE 5
Efficacy of various preservatives against P. aeruginosa.

| Conc. of ingredients in cream (% w/w) | Colony counts (CFU/gm) |
|---|---|
| Base(Control) | 5.0 × 10⁹ |
| 0.5% Octanediol | 1.6 × 10³ |
| 0.2% Lactic acid | 2.0 × 10⁶ |
| 0.2% Citric acid | 2.5 × 10⁶ |
| 0.5% Octanediol + 0.2% Lactic acid | 0 |
| 0.5% Octanediol + 0.2% Citric acid | 0 |

Conclusion: Octanediol in combination with organic acids exhibited enhanced activity against P. aeruginosa.

### Example 5

### Activity of Low Concentrated Symdiol™

Activity of lower concentration of Symdiol™ (0.2% w/w) with organic acids was measured. The cream base used in this study was supplied by Symrise Corporation. This base is an oil/water base.

Table 6 shows the concentration of preservative in the cream and effect of the preservative in the cream against 10⁷ cfu of test organism/gm of cream.

Method of evaluation: Method B was used for this test. Method B: Same as described in Method A except a higher inoculum was used. A 100 μL inoculum containing 10⁷ CFU of test organism was added to each 1 gm of cream and incubated at 37° C. Microbial growth measured as CFU was counted after 24 hour incubation at 37° C.

TABLE 6

| | Colony counts (CFU/gm) | | | |
|---|---|---|---|---|
| Conc. of ingredients | P. | | | |
| in cream (% w/w) | aeruginosa | S. aureus | E. coli | C. albicans |
| Base(Control) | 2.0 × 10⁸ | 2.0 × 10⁷ | 2.4 × 10⁸ | 1.0 × 10⁷ |
| 0.3% Symdiol ™ | 2.0 × 10⁶ | 1.0 × 10⁵ | 1.6 × 10⁴ | 1.5 × 10⁵ |
| 0.2% Lactic acid | 2.0 × 10⁶ | 6.0 × 10⁴ | 1.2 × 10⁶ | 2.0 × 10⁶ |
| 0.2% Citric acid | 1.0 × 10⁶ | 2.0 × 10⁴ | 2.0 × 10⁶ | 2.0 × 10⁶ |
| 0.3% Symdiol ™ + | 1.0 × 10⁵ | 5.0 × 10⁴ | 0 | 2.0 × 10⁴ |
| 0.2% Lactic acid | | | | |
| 0.3% Symdiol ™ + | 0 | 7.0 × 10⁴ | 0 | 1.0 × 10⁴ |
| 0.2% Citric acid | | | | |

Conclusion: The organic acids showed enhanced activity even with a lower concentration of Symdiol™.

### Example 6

### Enhanced Activity of Lower Concentration of Octanediol (0.2% w/w) when Combined with AHAs

The cream base used in this study was supplied by Symrise Corporation. This base is an oil/water base. The following table shows the concentration of preservative in the cream and the preservative-effect of these creams against 10⁷ CFU of test organism/gm of cream.

Method of evaluation: Method B was used for this test with C. albicans as test organism.

TABLE 7

| | Colony counts (CFU/gm) |
|---|---|
| Conc. of ingredients in cream (% w/w) | C. albicans |
| Base(Control) | 1.0 × 10⁷ |
| 0.2% Octanediol | 1.0 × 10⁵ |
| 0.2% Lactic acid | 2.0 × 10⁶ |
| 0.2% Citric acid | 2.0 × 10⁶ |
| 0.2% Octanediol + 0.2% Lactic acid | 2.0 × 10⁴ |
| 0.2% Octanediol + 0.2% Citric acid | 2.0 × 10³ |

Conclusion: Citric acid showed enhanced activity even with a lower concentration of octanediol.

Preservative Compositions

Development of Various Preservative Compositions for Use in Cosmetics and Skin Creams.

The following compositions were prepared and can be used at a range of 0.1-2.0% w/w as a preservative in topical creams and cosmetics to prevent spoilage due to growth of gram positive, gram negative bacteria as well as mold and fungi. These compositions in addition to preserving the products can also provide silky and smooth texture to the skin.

TABLE 8

| No. | Emollient Solvent (% w/w) | AHA (% w/w) | Other (% w/w) |
|---|---|---|---|
| 1 | 50% Octanediol | 20% Mandelic acid | 30% water |
| 2 | 50% Octanediol | 20% Lactic acid | 30% water |
| 3 | 50% Octanediol | 20% Citric acid | 30% water |
| 3A | 50% Octanediol | 10% Citric acid | 40% water |
| 3B | 30% Octanediol | 20% Citric acid | 30% water |
| 8 | 50% Symdiol ™ | 20% Citric acid | 30% water |
| 10 | 50% Symdiol ™ | 20% Lactic acid | 30% water |
| 12 | 50% Symdiol ™ | 20% Mandelic acid | 30% water |

Monoesters such as propylene heptanoate, propylene caprylate, glyceryl caprylate can also be used along with organic acids or in combination with other emollient solvents.

### Example 7

### Evaluation of Various Preservative Compositions

Evaluation of the effect of various preservative compositions incorporated in an oil/water base against C. albicans (yeast) incubated at 30° C. Yeast and fungi grow optimally at 30° C. and therefore the efficacy of the preservative was evaluated at this temperature.

Method of evaluation: Method C: Same as Method B except that incubation was at 30° C. and subculturing is done on Sabouraud agar.

The following preservatives at 1.0% and 0.5% concentrations were added to the cream. C. albicais at 10⁷ cfu/gm base was added to the base and incubated at 30° C. for 24 hours.

TABLE 9

| | C. albicans |
|---|---|
| Preservative in cream (% w/w) | (CFU/gm cream) |
| None (Control) | 1.5 × 10⁷ |
| 1.0% of preservative 2 (50% Octanediol + 20% | 0 |
| Lactic acid) | |
| 0.5% of preservative 2 (50% Octanediol + 20% | 6.5 × 10³ |
| Lactic acid) | |
| 1.0% of preservative 3 (50% Octanediol + 20% Citric | 0 |
| acid) | |
| 0.5% of preservative 3 (50% Octanediol + 20% Citric | 8.1 × 10³ |
| acid) | |
| 1.0% of preservative 3A (50% Octanediol + 10% | 0 |
| Citric acid) | |
| 1.0% of preservative 3B (30% Octanediol + 20% | 0 |
| Citric acid) | |
| 1.0% of preservative 8 (50% Symdiol ™ + 20% Citric | 8.4 × 10³ |
| acid) | |
| 0.5% of preservative 8 (50% Symdiol ™ + 20% Citric | 5.6 × 10⁴ |
| acid) | |
| 1.0% of preservative 10 (50% Symdiol ™ + 20% | 1.4 × 10³ |
| Lactic acid) | |
| 0.5% of preservative 10 (50% Symdiol ™ + 20% | 3.4 × 10⁴ |
| Lactic acid) | |
| 50% Octanediol | 0 |
| 30% Octanediol | 1.4 × 10⁵ |
| 50% Symdiol ™ | 3.9 × 10⁴ |
| 30% Symdiol ™ | 2.0 × 10⁶ |
| 20% Lactic acid | 5 × 10⁶ |
| 20% Citric acid | 2.5 × 10⁶ |
| 0.12% Lactic acid | 4.2 × 10⁶ |

Conclusions: Enhanced activity is observed in this experiment. All the preservative compositions significantly reduced C. albicans growth. Octanediol was superior to Symdiol™.

### Example 8

### Measurement of Efficacy of Preservative Compositions

Efficacy of preservatives incorporated in the oil/water base and inoculated with the yeast C. albicans and fungus Aspergillus niger.

The cream base and the cream containing the preservatives were inoculated with 107 cfu of C. albicans/gm and 105 cfu of Aspergillus niger/gm; and incubated at 30° C. for 24 hours and 48 hours.

Method of Evaluation: Testing was done using Method C.

TABLE 10

| | Growth of C. albicans and | | | |
|---|---|---|---|---|
| | A. niger (CFU/gm cream) | | | |
| Preservative in cream | C. albicans | | Aspergillus niger | |
| (% w/w) | 24 h | 48 h | 24 h | 48 h |
| None (Control) | 1.5 × 10⁷ | 3.0 × 10⁷ | 2.0 × 10⁵ | 5.0 × 10⁵ |
| 1.0% of preservative 3 | 0 | 0 | 3.0 × 10¹ | 0 |
| 0.6% of preservative 3 | 0 | 0 | 5.0 × 10² | 5.0 × 10¹ |
| 1.0% of preservative 8 | 8.4 × 10³ | 0 | 8.0 × 10² | 0 |

Conclusion: Both the preservative groups showed significant reduction in the growth as compared to the control. Preservative 3 was more effective than Preservative 7.

### Example 9

### Effect of Preservatives in Cream Base

Efficacy of preservatives in a creaminoculated with a higher concentration of Aspergillus niger (107 efu/gm).

TABLE 11

| | |
|---|---|
| | Growth (CFU/gm cream) |
| Preservative in cream | Colony counts in the cream |

| (% w/w) | 24 hr | 48 hr |
|---|---|---|
| None ( Control) | 1.5 × 10⁷ | 1.0 × 10⁷ |
| 1.0% of preservative 3 | 0 | 0 |
| 1.0% of preservative 7 | 8.5 × 10² | 0 |

### Example 10

### Effect of Various Preservative Systems on Bacteria Growth

Effect of various preservative systems on the growth of bacteria inoculated in the cream at a concentration of 10⁷ cfu/gm of cream and incubated at 37° C.

TABLE 12

| | Growth of Organism (CFU/gm) | | |
|---|---|---|---|
| Preservative (% w/w) | P. aeruginosa | S. aureus | E. coli |
| None (control) | 5.0 × 10⁹ | 3.0 × 10⁸ | 4.8 × 10⁸ |
| 1.0% of preservative 2 | 0 | 0 | 0 |
| 0.5% of preservative 2 | 1.0 × 10⁴ | 2.0 × 10² | 3.4 × 10³ |
| 1.0% of preservative 3 | 0 | 0 | 0 |
| 0.5% of preservative 3 | 3.0 × 10⁴ | 0 | 2.8 × 10³ |
| 1.0% of preservative 8 | 0 | 0 | 0 |
| 0.5% of preservative 8 | 1.0 × 10² | 5.3 × 10⁴ | 6.0 × 10⁴ |

Conclusion: All of the preservatives tested completely inactivated the bacteria at 1.0% concentration and showed significant reduction at 0.5% concentration.

### Example 11

### Efficacy of Preservatives

Efficacy of preservatives containing an emollient solvent and AHA against Aspergillus niger. The following preservative compositions were prepared (% w/w):

TABLE 13

| | |
|---|---|
| J | 50% Octanediol + 30% citric acid + 20% water |
| P | 50% Octanediol + 30% lactic acid + 20% water |
| R | 50% Octanediol + 50% lactic acid |
| U | 50% Sensiva ™ + 50% lactic acid |
| V | 50% Sensiva ™ + 30% citric acid + 20% water |

1.0% w/w of each above preservative was added to the cream base, which was then inoculated with 1×10⁶ cfu/gm of A. niger., and incubated at 30° C. for 72 hours, and tested as described in method A.

TABLE 14

| | Growth of organism (CFU/gm) |
|---|---|
| Preservative (w/w) | (72 hours incubation in cream) |
| Base (control) | 3 × 10⁶ |
| Composition J | 6 × 10¹ |
| Composition P | 8 × 10¹ |
| Composition R | 4 × 10¹ |
| Composition U | 8 × 10¹ |
| Composition V | 1 × 10² |

Conclusion: Significant reduction in the growth was observed in all the groups.

Preservative compositions containing emollient solvents such as octanediol or Symdiol™ or Sensiva™ or monoesters in combination with AHAs (metal salts of these acids are excluded) can be used for preventing microbial growth and spoilage in cosmetic and topical skin formulations with the added benefit of providing silky smooth texture. These preservative compositions are odorless and colorless and small concentrations (0.3-1% w/w) can be added to cosmetic/topical formulations to prevent bacterial, yeast and fungal growth within 24-48 hours after contamination.

### Example 12

### Compatibility of Emollient-Alpha Hydroxy Acid Containing Systems

(E-AHA systems) with hydrophilic and hydrophobic products. The following compositions containing Octanediol and Alpha hydroxy acids were prepared for use in various topical and cosmetic products; they were added to hydrophilic topical creams and hydrophobic silicone emulsions at 1% concentrations. All formulations are % w/w. All of them were compatible in both products except S-17 which was not compatible with hydrophobic products.

TABLE 15

| | |
|---|---|
| D-2 | 50% Octanediol + 40% Sensiva + 10% Lactic acid |
| F | 50% Octanediol + 20% Mandelic acid + 30% Ethanol (95%) |
| G-2 | 50% Octanediol + 1% Malic acid + 49% Propylene Glycol |
| H-2 | 99% Octanediol + 1% Malic acid |
| J-2 | 50% Octanediol + 49% Ethanol + 1% Malic acid |
| L-2 | 60% Octanediol + 39% Ethanol + 1% Malic acid |
| M-2 | 60% Octanediol + 38% Ethanol + 2% Malic acid |
| N-2 | 60% Octanediol + 38% Ethanol + 2% Lactic acid |
| O-2 | 60% Octanediol + 37% Ethanol + 3% Lactic acid |
| P-2 | 60% Octanediol + 36% Ethanol + 4% Lactic acid |
| Q-2 | 60% Octanediol + 37% Sensiva + 3% Lactic acid |
| R-2 | 60% Octanediol + 39% Ethanol + 1% Lactic acid |
| S-2 | 60% Octanediol + 39% Ethanol + 1% Glycolic acid |
| S-3 | 60% Octanediol + 38% Ethanol + 2% Glycolic acid |
| S-4 | 60% Octanediol + 37% Ethanol + 3% Glycolic acid |
| S-5 | 60% Octanediol + 37% Sensiva + 3% Glycolic acid |
| S-6 | 60% Octanediol + 39% Sensiva + 1% Glycolic acid |
| S-8 | 60% Octanediol + 40% Lactic acid |
| S-7 | 60% Octanediol + 39% Sensiva + 1% Lactic acid |
| S-9 | 50% Octanediol + 30% Sensiva + 20% Lactic acid |
| S-10 | 55% Octanediol + 40% Sensiva + 5% Glycolic acid |
| S-11 | 80% Octanediol + 20% Lactic acid |
| S-12 | 60% Octanediol + 35% Sensiva + 5% Lactic acid |
| S-13 | 60% Octanediol + 35% Propylene glycol + 5% Lactic acid |
| S-14 | 95% Octanediol + 5% Glycolic acid |
| S-15 | 70% Octanediol + 30% Lactic acid |
| S-16 | 70% Octanediol + 30% Glycolic acid |
| S-17 | 50% Octanediol + 10% Lactic acid + 20% Citric acid + 20% |
| | water |
| S-20 | 99% Octanediol + 1% Lactic acid |
| PS-2 | 80% Octanediol + 20% Glycolic acid |
| PS-3 | 50% Octanediol + 50% Glycolic acid |
| PS-4 | 50% Octanediol + 50% Lactic acid |
| PS-6 | 50% Octanediol + 30% Sensiva + 20% Glycolic acid |
| PS-7 | 40% Octanediol + 25% Sensiva + 25% Farnesol + 10% Lactic |
| | acid |
| PS-8 | 60% Octanediol + 40% Lactic acid |
| PS-9 | 30% Octanediol + 30% Sensiva + 40% Lactic acid |
| PS-10 | 95% Octanediol + 5% Lactic acid |

### Example 13

Selection of the Best E-AHA Composition for Use in Hydrophilic and Hydrophobic Products to Prevent the Growth of Gram Positive and Gram Negative Bacteria, Fungi and Yeast

It was found that certain compositions containing Octanediol and Lactic acid (Oct-Lac) were highly effective in preventing microbial growth in both Hydrophobic and Hydrophilic systems and are also compatible. The following compositions were added to various Hydrophilic creams and Silicon emulsions and tested for their efficacy in preventing microbial growth for long term. All components are % w/w.

TABLE 16

| | |
|---|---|
| Q2 | 60% Octanediol + 37% Sensiva + 3% Lactic acid |
| S11 | 80% Octanediol + 20% Lactic acid |
| S15 | 70% Octanediol + 30% Lactic acid |
| S20 | 99% Octanediol + 1% Lactic acid |
| PS4 | 50% Octanediol + 50% Lactic acid |
| PS8 | 60% Octanediol + 40% Lactic acid |
| PS10 | 95% Octanediol + 5% Lactic acid |

### Example 14

### Microbial Growth in a Hydrophilic Moisturizing Cream (Johnson and Johnson) Containing 1.0% Oct-Lac 7 Days Post Contamination

Method A: The following microbial cultures used for contamination.

Bacteria: An overnight culture of bacteria grown in Trypticase Soy Broth (TSB) is diluted with TSB to obtain 10⁸ CFU organism/ml.

Yeast (C. Albicans): An overnight culture of C. albicans grown in Sabaraud Dextrose Broth is diluted to 10⁶ cfu organism/ml.

Fungi (Aspergillus niger): A pure inoculum suspension was prepared by rinsing the fungi slant (incubated for 48 hours after inoculation) with sterile water and vortexed to get a uniform suspension. This suspension was diluted with sterile water to obtain 10⁶ cfu organism/ml.

For the test samples, 1% of the Oct-Lac was added to 10 grams of the cream. From this sample, 1 gram aliquots were placed into 10 ml sterile plastic culture tubes and 0.1 ml (100 microliters) of the test inoculun was added and vortexed until uniformly blended. The tubes were then placed into incubators under the following temperatures: 30° C. for Aspergillus niger and 37° C. for the remaining three microbes. All tubes were incubated for a total of 7 days.

At the end of the incubation period 9.0 ml of Butterfield Phosphate Buffered solution with neutralizer was added the incubated cultured sample and vortexed until completely mixed. The samples were serially diluted and then plated in Trypticase soy agar (TSA) in the case of Bacteria and Sabaraud dextrose agar (SDA) in the case of Fungi and Yeast; the plates were incubated at 37° C. temperature in the case of bacteria and yeast and 30° C. in the case of Fungi for 24-48 hours and the counts were read.

Results.

TABLE 17
Growth (CFU/gm)

| | Control | PS-8 | PS-10 | S-20 | S-11 |
|---|---|---|---|---|---|
| S. aureus | 7.5 × 10⁶ | 0 | 0 | 0 | 0 |
| E. coli | 1.0 × 10⁷ | 0 | 0 | 0 | 0 |
| A. niger | 4.9 × 10⁴ | 93 | 0 | 2.0 × 10³ | 0 |
| C. albicans | 3.8 × 10⁵ | 0 | 0 | 33 | 0 |

Conclusion: All the groups were highly effective except S-20 which contains only 1% Lactic acid showed 1.0 log reduction against A. niger.

### Example 15

### Microbial Growth in J&J Hydrophilic Cream-1 Containing PS-8 Post Contamination with Bacteria, Yeast and Fungi

Method B. Same as Method A except 2% of Oct-Lac was added to the cream.

TABLE 18
Microbial Counts (CFU)/gm cream (1
and 3 Days Post-contamination)

| | |
|---|---|
| Placebo (control) | Cream + PS 8 |

Day

| Organism | 1 Day | 3 Day | 1 Day | 3 |
|---|---|---|---|---|
| S. aureus | 2.7 × 10⁴ | 2.6 × 10⁴ | 0 | 0 |
| (ATCC#6538) | | | | |
| E. coli | 1.4 × 10⁴ | 1.6 × 10⁴ | 0 | 0 |
| (ATCC#35218) | | | | |
| P. aeruginosa | 6.6 × 10⁴ | 6.1 × 10⁴ | 0 | 0 |
| (ATCC# 15442) | | | | |
| Aspergillus niger | 4.6 × 10³ | 2.2 × 10³ | 97 | 0 |
| (ATCC#16404) | | | | |
| C. albicans | 1.3 × 10⁴ | 1.0 × 10⁴ | 0 | 0 |
| (ATCC#10231) | | | | |

Conclusion: The cream without the Oct-Lac showed microbial counts 24 hours after inoculation. The cream with Oct-Lac (S-8) had negligible microbial counts after 24 hours or 3 days.

### Example 16

### Microbial Growth in J&J Hydrophilic Cream-1 Containing S-11 Post Contamination with Bacteria, Yeast and Fungi

The test procedure used here is the same as Method B.

TABLE 19
Microbial Counts (CFU)/gm cream
(1 and 3, 4 Days Post-Contamination)

| | Placebo (control) | | | Cream + S-11 | | |
|---|---|---|---|---|---|---|
| Organism | 1 Day | 3 Day | 4 Day | 1 Day | 3 Day | 4 Day |

| | | | | | | |
|---|---|---|---|---|---|---|
| S. aureus | 2.4 × 10⁶ | 2.2 × 10⁶ | 2.0 × 10⁶ | 0 | 0 | 0 |
| (ATCC#6538) | | | | | | |
| E. coli | 1.0 × 10⁷ | 1.1 × 10⁷ | 1.2 × 10⁷ | 0 | 0 | 0 |
| (ATCC#35218) | | | | | | |
| P. aeruginosa | 8.3 × 10⁷ | 8.2 × 10⁷ | 8.0 × 10⁷ | 0 | 0 | 0 |
| (ATCC# 15442) | | | | | | |
| Aspergillus niger | 3.3 × 10⁴ | 3.0 × 10⁴ | 3.0 × 10⁴ | 1.2 × 10² | 1.0 × 10² | 23 |
| (ATCC#16404) | | | | | | |
| C. albicans | 3.5 × 10⁵ | 3.2 × 10⁵ | 3.3 × 10⁵ | 0 | 0 | 0 |
| (ATCC#10231) | | | | | | |

Conclusion: The cream without the Oct-Lac showed microbial counts 24 hours after inoculation. The cream with the Oct-Lac system (S-11) significantly lower microbial counts after 24 hours and 4 days.

### Example 17

### Microbial Growth in DC Silicones Containing Oct-Lac Composition (Q2) Post Contamination with Bacteria, Yeast and Fungi

The test procedure used here is the same as Method B.

TABLE 20
Microbial counts (CFU)/gm silicone (7 days post inoculation)

| | | | Aspergillus | |
|---|---|---|---|---|
| Silicone elastomer | S. aureus | E. coli | niger | C. albicans |
| Silicone-1 (control) | >10⁶ | >10⁶ | 1.3 × 10⁵ | 2.6 × 10⁶ |
| Silicone 1 + Q2 | 0 | 0 | 0 | 0 |
| Silicone-2 (control) | >10⁶ | >10⁶ | 2.0 × 10⁵ | 2.6 × 10⁶ |
| Silicone 2 + Q2 | 20 | 0 | 0 | 0 |

Conclusion: Hydrophobic Silicone without the Oct-Lac showed microbial counts seven days after inoculation. The cream with the Oct-Lac system (Q-2) had negligible microbial counts.

### Example 18

### Microbial Growth in DC Silicones Containing Oct-Lac Composition (Q2) Post Contamination with Bacteria, Yeast and Fungi

The test procedure used here is the same as Method B.

TABLE 21
Microbial counts (CFU)/gm silicone (14 days post Contamination)

| | | | Aspergillus | |
|---|---|---|---|---|
| Silicone elastomer | S. aureus | E. coli | niger | C. albicans |
| Silicone-1 (control) | 2.0 × 10⁶ | 1.0 × 10⁶ | 1.0 × 10⁵ | 2.0 × 10⁶ |
| Silicone 1 + S-11 | 0 | 0 | 0 | 0 |
| Silicone-2 (control) | 1.5 × 10⁵ | 1.8 × 10⁶ | 2.5 × 10⁴ | 1.6 × 10⁶ |
| Silicone 2 + S-11 | 0 | 0 | 0 | 0 |

Conclusion: Hydrophobic Silicone without the Oct-Lac showed microbial counts 14 days after inoculation. The cream with the Oct-Lac system (S-11) had no microbial counts.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, procedures, and the like are cited throughout this application, the disclosures of which are incorporated herein by reference in their entireties. Various publications are cited herein, the contents of which are hereby incorporated herein in their entireties by reference.

1. A topical composition comprising an antimicrobial preservative composition wherein the antimicrobial preservative composition comprises a combination of emollient solvent and an alpha hydroxy acid, and wherein the combination shows greater than additive antimicrobial activity.

2. The topical composition of claim 1, further comprising an essential oil component.

3. The topical composition of claim 1, comprising a combination of emollient solvent and alpha hydroxy acid showing greater than additive antimicrobial activity in an amount effective in reducing or preventing the growth of microorganisms.

4. The topical composition of claim 1, comprising a combination of emollient solvent and alpha hydroxy acid showing greater than additive antimocrobial activity in an amount having broad spectrum antimicrobial activity.

5. The topical composition of claim 1, wherein the broad spectrum antimicrobial activity comprises activity against bacteria, yeast-like fungi or non-yeast fungi or viruses.

6. The topical composition of claim 1, wherein the emollient solvent is selected from the group consisting of octanediol, hexanediol, Symdiol™, Sensiva™ (octoxy glycerin), propylene hepanoate, propylene caprylate and glyceryl caprylate.

7. The topical composition of claim 1, wherein the alpha hydroxy organic acid is selected from the group consisting of citric, lactic, benzoic, glycolic, mandelic, malic and tartaric acid.

8. The topical composition of claim 2, wherein the essential oil component is selected from the group consisting of thyme oil, savory oil, oregano oil, rosewood oil, lavendar oil, basil oil, farnesol, and bisbolol.

9. A topical composition comprising an antimicrobial preservative composition wherein the antimicrobial preservative composition consists of a synergistic combination of one or more emollient solvent, an alpha hydroxy acid, an optionally an essential oil component.

10. A topical composition comprising:
from about 0.3% to about 5% of about 50% to about 99% (w/w) stock solution of an emollient solvent; and
from about 1% to about 50% alpha hydroxy acids (w/w).

11. The topical composition of claim 1, wherein the emollient solvent is present in amounts ranging from about 0.05% to about 5% w/w.

12. The topical composition of claim 11, wherein the emollient solvent is present in amounts ranging from about 0.05% to about 1% w/w.

13. The topical composition of claim 1, wherein the AHA is present in amounts ranging from about 0.01% to about 5.0% w/w.

14. The topical composition of claim 13, wherein the AHA is present in amounts ranging from about 0.05% to about 3% w/w.

15. The topical composition of claim 2, wherein the essential oil component is present in an amount ranging from about 0.01% and 10% w/w.

16. The topical composition of claim 15, wherein the essential oil component is present in an amount ranging from about 0.05% to about 1.0% w/w.

17. A topical composition comprising: about 60% octanediol, about 37% sensiva, and about 3% lactic acid.

18. A topical composition comprising: about 80% octaneiol and about 20% lactic acid.
